(19) United States
(12) Patent Application Publication
(10) Pub. No.: US 2011/0237770 A1
(43) Pub. Date: Sep. 29, 2011

(54) VISCOSITY REDUCING AGENTS FOR POLYETHER POLYOLS

(76) Inventors: Peter Daute, Beverstedt (DE); Bernhard Bartnick, Monheim (DE)

(21) Appl. No.: 13/060,302

(22) PCT Filed: Aug. 28, 2009
(86) PCT No.: PCT/EP2009/061122

§ 371 Date: Jun. 17, 2011

(30) Foreign Application Priority Data
Aug. 28, 2008 (DE) 10 2008 044 706.4

(51) Int. Cl. C08G 18/08; C07C 69/708; B29C 45/00

(52) U.S. Cl. 528/76; 560/186; 264/328.1

(57) ABSTRACT

The present invention relates to a process for the preparation of a polyurethane, comprising the process steps:
i) provision of a polyisocyanate component comprising at least one polyisocyanate;
ii) provision of a polyol component comprising at least one polyether polyol, one polyester polyol or a mixture of a polyether polyol and a polyester polyol, wherein the polyol component comprises a polyol ester of a polyol and a monocarboxylic acid;
iii) bringing of the polyisocyanate component into contact with the polyol component to form a polyurethane.

This application is a national stage application under 35 U.S.C. 371 of International Application No. PCT/EP2009/061122 filed 28 Aug. 2009, which claims priority to German Application No. DE 10 2008 044 706.4 filed 28 Aug. 2008, the disclosures of which are expressly incorporated herein by reference.

The present invention relates to a process for the preparation of a polyurethane, the polyurethane obtainable by this process, a polyol component comprising a polyether polyol, a polyester polyol or a mixture of a polyether polyol and a polyester polyol, a process for the preparation of a polyol component comprising a polyether polyol, a polyester polyol or a mixture of a polyether polyol and a polyester polyol, the polyol component comprising a polyether polyol, a polyester polyol or a mixture of a polyether polyol and a polyester polyol obtainable by this process, the use of this polyol component comprising a polyether polyol, a polyester polyol or a mixture of a polyether polyol and a polyester polyol, and the use of a polyol ester.

### BACKGROUND

Polyurethanes have been known for a long time and are described in several instances. Depending on the nature of the starting components employed for the preparation of the polyurethane, they can be in the form of a foamed or non-foamed plastic. If the plastic is foamed, this can in turn be in the form of a permanently elastic flexible foam, which is suitable, for example, for the production of sports shoe soles or mattresses for sleeping on, or in the form of a rigid foam, which can be employed, for example, as assembly foam. An overview of the possible uses of polyurethanes is given, for example, by Reinhard Leppkes in “Polyurethane—Werkstoff mit vielen Gesichtern”, 5th edition. Verlag Moderne Industrie, 2003.

The preparation of polyurethanes is also adequately known from the prior art. This is conventionally carried out by reaction of polyisocyanates, diphenylmethane-diisocyanate (MDI) and, in particular, mixtures of diphenylmethane-diisocyanate and the higher homologues polyphenylene-polymethylenepolyisocyanates (crude MDI) usually being employed here, with compounds having at least two hydrogen atoms which are reactive with isocyanate groups. A comprehensive overview of the production and use of rigid polyurethane foams is to be found, for example, in Kunststoff-Handbuch, volume 7, Polyurethane, 1st edition 1966, published by Dr. R. Vieweg and Dr. A. Höchtlen, 2nd edition 1983, published by Dr. Günter Oertel, and 3rd edition 1993, published by Dr. Günter Oertel, Carl Hanser Verlag, Munich, Vienna.

### SUMMARY

The present invention includes various embodiments as set forth herein.

Polyols, in particular polyether polyols and polyester polyols, are often employed as the compound having at least two hydrogen atoms which are reactive with isocyanate groups both in the production of rigid foams and in the production of flexible foams, the polyether polyols being obtainable by reaction of alkylene oxides, for example ethylene oxide or propylene oxide, with starter molecules, such as, for example, water, amines or alcohols, while the polyester polyols are conventionally obtained by condensation of polyfunctional alcohols with polyfunctional carboxylic acids. Processes for the preparation of polyether and polyester polyols are described, for example, in WO-A-2008/084054. However, the polyester or polyether polyols obtained in this way as a rule have a very high viscosity, so that they can be mixed only very poorly with the polyisocyanates. If the polyol component and the polyisocyanate component cannot be mixed with one another homogeneously enough, however, this also has disadvantages for the resulting polyurethane.

A further disadvantage of the processes known from the prior art for the preparation of polyurethanes based on polyether or polyester polyols is that the reaction mixture obtained by mixing the polyol component with the polyisocyanate component also has a comparatively high viscosity, which makes its use in so-called reaction injection molding processes (“RIM processes” for short) difficult, especially if cavities of small volume have to be filled.

### DETAILED DESCRIPTION

The present invention was based on the object of overcoming the disadvantages resulting from the prior art in connection with the preparation of polyurethanes from polyisocyanates and polyether or polyester polyols.

In particular, the present invention was based on the object of providing a process for the preparation of a polyurethane based on polyisocyanates and polyether or polyester polyols, with the aid of which these components can be mixed with one another more easily.

The present invention was furthermore based on the object of providing a process for the preparation of a polyurethane based on polyisocyanates and polyether or polyester polyols, with the aid of which polyurethanes with improved product properties compared with corresponding polyurethanes obtainable from the prior art by conventional processes can be obtained.

The present invention was also based on the object of providing a process for the preparation of a polyurethane based on polyisocyanates and polyether or polyester polyols, which is also suitable in particular for the production of shaped articles of small volume or shaped articles comprising sections of small volume.

A contribution towards achieving the abovementioned objects is made by a process for the preparation of a polyurethane comprising the process steps:
i) providing a polyisocyanate component comprising at least one polyisocyanate;
ii) providing a polyol component comprising at least one polyether polyol, one polyester polyol or a mixture of a polyether polyol and a polyester polyol, wherein the polyol component comprises a polyol ester of a polyol and a monocarboxylic acid;
iii) bringing of the polyisocyanate component into contact with the polyol component to form a polyurethane.

In process step i) of the process according to the invention, a polyisocyanate component comprising at least one polyisocyanate is first provided.

In this context, possible polyisocyanates are all the polyisocyanates known to the person skilled in the art for the preparation of polyurethanes, which can optionally also be employed as a mixture comprising at least two structurally different polyisocyanates. In this context, either aliphatic isocyanates, such as hexamethylenediisocyanate (HDI) or isophorone-diisocyanate (IPDI), or, preferably, aromatic isocyanates, such as toluoylene-diisocyanate (TDI), diphenylmethane-diisocyanate (MDI) or mixtures of diphenylmethane-diisocyanate and polymethylenepolyphenylene-polyisocyanates (crude MDI), can be used. It is also possible to employ isocyanates which have been modified by incorporation of urethane, uretdione, isocyanurate, allophanate, uretonimine and other groups, so-called modified isocyanates.

Polyisocyanate prepolymers can furthermore also be employed as the polyisocyanate component. These prepolymers are known in the prior art. The preparation of such polyisocyanate prepolymers is carried out in a manner known per se by reacting the polyisocyanates described above, for example at temperatures of about 80° C., with, for example, polyether polyols or polyester polyols, but in particular with the polyol component described below, to give the prepolymer. The polyol-polyisocyanate ratio is in general chosen such that the NCO content of the prepolymer is 8 to 25 wt. %, preferably 10 to 24 wt. %, particularly preferably 13 to 23 wt. %.

The polyisocyanate component can optionally also comprise, in addition to the polyisocyanate described above, one of the reactive components described in EP-A-0 477 638, for example one of the epoxide components described in this prior art, reference being made to the disclosure content of EP-A-0 477 638 with respect to the nature of the epoxides, the amount in which these are employed and with respect to the nature and manner of the pretreatment of the polyisocyanate component with the epoxide component.

In process step ii) of the process according to the invention, a polyol component comprising at least one polyether polyol, one polyester polyol or a mixture of a polyether polyol and a polyester polyol, wherein the polyol component comprises a polyol ester of a polyol and a monocarboxylic acid as a viscosity reducer of the polyol component, is provided.

In this context, the preparation of this polyol component is preferably carried out by mixing a polyether polyol component, a polyester polyol component or a mixture of a polyether polyol component and a polyester polyol component with the polyol ester. In this connection, it is preferable in particular for the polyether polyol component, the polyester polyol component or the mixture of the polyether polyol component and the polyester polyol component to be based on a polyether polyol, a polyester polyol or a mixture of a polyether polyol and a polyester polyol, or to consist of this, to the extent of at least 50 wt. %, still more preferably to the extent of at least 60 wt. %, moreover preferably to the extent of at least 75 wt. %, moreover still more preferably to the extent of at least 95 wt. % and most preferably to the extent of at least 99 wt. %, in each case based on the total weight of the polyether polyol component, the polyester polyol component or the mixture of the polyether polyol component and the polyester polyol component.

In this connection, it is furthermore particularly preferable according to the invention for the polyether polyol component employed for the preparation of the polyol component to be a high-viscosity polyether polyol component, which preferably has a viscosity determined by the Brookfield method at 25° C. of at least 500 mPas, particularly preferably of at least 1,000 mPas, and most preferably of at least 2,000 mPas, where the viscosity determined by the Brookfield method at 25° C. is preferably in a range of from 500 to 12,000 mPas, still more preferably in a range of from 1,000 to 10,000 mPas and most preferably in a range of from 2,000 to 8,000 mPas. If a polyester polyol component is employed for the preparation of the polyol component, it is particularly preferable according to the invention for the polyester polyol component employed for the preparation of the polyol component likewise to be a high-viscosity polyester polyol component, which preferably has a viscosity determined by the Brookfield method at 25° C. of at least 1,000 mPas, particularly preferably of at least 2,000 mPas, and most preferably of at least 4,000 mPas, where the viscosity determined by the Brookfield method at 25° C. is preferably in a range of from 1,000 to 20,000 mPas, still more preferably in a range of from 2,000 to 15,000 mPas and most preferably in a range of from 4,000 to 10,000 mPas.

The polyether polyols contained in the polyol component or the polyether polyol component employed for the preparation of this polyol component are preferably obtainable by reaction of an alkylene oxide with water, an amine, an amino alcohol or an alcohol as the starter molecule, where, for example, tetrahydrofuran, ethylene oxide, 1,2-propylene oxide, 1,3-propylene oxide, 1,2- or 2,3-butylene oxide or styrene oxide can be employed as the alkylene oxide, but 1,2-propylene oxide or ethylene oxide are particularly preferably employed. The alkylene oxides can be used individually, in alternation successively or as mixtures. The use of an ethylene oxide/propylene oxide mixture leads, for example, to a polyether polyol with random distribution of the ethylene oxide/propylene oxide units. However, it is also possible first to employ an ethylene oxide/propylene oxide mixture and then to use only propylene oxide or ethylene oxide further before discontinuation of the polymerization, in order to obtain polyether polyols with a propylene oxide end cap or an ethylene oxide end cap in a targeted manner. In this connection, it is preferably in particular for the alkylene oxide content, particularly preferably the ethylene oxide or propylene oxide content, to be more than 50 wt. %, based on 100 percent by weight of alkylene oxides and starter molecule.

In general, in this context the polyether polyols are prepared by known processes, for example from one or more alkylene oxides, preferably from 1,2-propylene oxide and ethylene oxide, by anionic polymerization with alkali metal hydroxides, such as sodium or potassium hydroxide, or alkali metal alcoholates, such as sodium methylate, sodium or potassium ethylate or potassium isopropylate, as catalysts and with addition of the starter molecule, or by cationic polymerization with Lewis acids, such as antimony pentachloride, boron fluoride etherate and the like, or bleaching earth as catalysts.

In the case of the use of an alcohol as the starter molecule for the preparation of the polyether polyol, it is preferable according to the invention for the alcohol to be an alcohol having at least 2 hydroxyl groups in the molecule, preferably having 3 to 6 hydroxyl groups in the molecule. In this connection, particularly preferred dihydric alcohols are ethylene glycol, propylene glycol or butanediols, while preferred trihydric alcohols include, for example, glycerol, trimethylolpropane or castor oil or pentaerythritol. Preferred higher-hydric alcohols are, in particular, sugar alcohols, for example sucrose, glucose or sorbitol.

In the case of the use of an amine as the starter molecule for the preparation of the polyether polyol, it is preferable according to the invention for the amine to be an amine having at least two primary amino groups in the molecule. Examples of suitable aminic starter molecules which may be mentioned are, in particular, amines chosen from the group consisting of phenylenediamine, 2,3-toluoylenediamine, 2,4-toluoylenediamine, 3,4-toluoylenediamine, 2,6-toluoylenediamine, 4,4′-diaminodiphenylmethane, 2,4′-diaminodiphenylmethane, 2,2′-diaminodiphenylmethane, 1,2-ethylenediamine, 1,3-propylenediamine, 1,4-butylenediamine, 1,6-hexylenediamine, 1,8-octylenediamine, diethylenetriamine and dipropylenetriamine.

If amino alcohols are employed as the starter molecule, the use of monoethanolamine, diethanolamine or triethanolamine is possible here in particular.

Preferably, the polyether polyols employed in the process according to the invention have a functionality in a range of from preferably 2 to 8, particularly preferably from 3 to 8. It is furthermore preferable according to the invention for the polyether polyols to have hydroxyl numbers in a range of from 10 mg of KOH/g to 1,200 mg of KOH/g, particularly preferably in a range of from 50 mg of KOH/g to 800 mg of KOH/g and moreover preferably in a range of from 100 mg of KOH/g to 500 mg of KOH/g.

The polyether polyols employed in the process according to the invention are furthermore preferably characterized by a number-average molecular weight in a range of from 100 to 10,000 g/mol, particularly preferably in a range of from 200 to 5,000 g/mol and most preferably in a range of from 500 to 2,500 g/mol.

The polyether polyols employed in the process according to the invention can also optionally be modified still further, for example by catalytic adding on of carbon dioxide and alkylene oxides to form polyether-carbonate polyols, as is described, for example, in WO-A-2008/058913.

Example of polyether polyols which are suitable according to the invention or of polyether polyol components which are suitable for the preparation of the polyol component are, in particular, the polyether polyols of the Lupranol® brands of BASF AG, which are built up from recurring propylene oxide and/or ethylene oxide units. Further suitable homo-polyethylene oxides are, for example, the Pluriol® E brands of BASF AG, while suitable homo-polypropylene oxides include, for example, the Pluriol® P brands of BASF AG. Suitable mixed copolymers of ethylene oxide and propylene oxide are, for example, the Pluriol® PE or Pluriol® RPE brands of BASF AG. The products of PCC Rokita SA, Poland marketed under the Rokopol® brands, for example, can also be employed.

The polyester polyols contained in the polyol component or the polyester polyol component employed for the preparation of this polyol component are preferably obtainable by condensation of polyfunctional, preferably difunctional, alcohols having 2 to 12 carbon atoms, preferably 2 to 6 carbon atoms, with polyfunctional carboxylic acids having 2 to 12 carbon atoms, preferably with dicarboxylic acids.

Possible dicarboxylic acids are, for example: succinic acid, glutaric acid, adipic acid, suberic acid, azelaic acid, sebacic acid, decanedicarboxylic acid, maleic acid, fumaric acid, phthalic acid, isophthalic acid, terephthalic acid and the isomeric naphthalenedicarboxylic acids. Adipic acid is preferably employed. In this context, the dicarboxylic acids can be used both individually and in a mixture with one another. Instead of the free dicarboxylic acids, it is also possible to employ the corresponding dicarboxylic acid derivatives, such as e.g. dicarboxylic acid esters of alcohols having 1 to 4 carbon atoms or dicarboxylic acid anhydrides. Examples of alcohols which are dihydric and more than dihydric, in particular diols, are: ethanediol, diethylene glycol, 1,2- and 1,3-propanediol, dipropylene glycol, 1,4-butanediol, 1,5-pentanediol, 1,6-hexanediol, 1,10-decanediol, glycerol and trimethylolpropane. Ethanediol, diethylene glycol, 1,4-butanediol, 1,5-pentanediol, 1,6-hexanediol or mixtures of at least two of the diols mentioned, in particular mixtures of 1,4-butanediol, 1,5-pentanediol and 1,6-hexanediol, are preferably used. Polyester polyols from lactones, for example ε-caprolactone, or hydroxycarboxylic acids, e.g. w-hydroxycaproic acid and hydroxybenzoic acids, can furthermore be employed. Dipropylene glycol is preferably employed.

Concrete examples of suitable polyester polyols include, in particular, the Desmophen® polyesters obtainable from Bayer AG, for example Desmophen®650 MPA, Desmophen®651 MPA, Desmophen®670, Desmophen®670 BA and Desmophen®680X.

The hydroxyl number of the polyester alcohols is preferably in the range between 20 and 500 mg of KOH/g, particularly preferably between 40 and 100 mg of KOH/g.

It is now preferable according to the invention for the polyol component comprising the polyether polyol, the polyester polyol or the mixture of the polyether polyol and the polyester polyol to comprise a polyol ester of a polyol and a monocarboxylic acid. It has in fact been found, surprisingly, that in particular polyol esters of short-chain monocarboxylic acids can be employed as viscosity reducers for high-viscosity polyether polyols without these polyol esters having an adverse effect on polyurethane formation. In the process according to the invention, the preferably high-viscosity polyether polyol component employed, the preferably high-viscosity polyester polyol component employed or the preferably high-viscosity mixture of the polyether polyol component and the polyester polyol component is first brought into contact with the polyol ester, preferably by simple mixing, in order to reduce the viscosity of this polyol component. Only then is the polyol component obtained in this way brought into contact with the further components (polyisocyanate component and optionally further compounds having at least two hydrogen atoms which are reactive with isocyanate groups and optionally further additives) to form the polyurethane. However, it is also conceivable to add further additives, in particular fillers, already to the polyol component and to mix the still more viscous polyol component obtained in this way with the polyol ester.

According to a preferred embodiment of the process according to the invention, the polyol component comprises the polyol ester in an amount in a range of from 0.1 to 30 wt. %, particularly preferably in a range of from 1 to 20 wt. % and most preferably in a range of from 5 to 15 wt. %, in each case based on the total weight of the polyol component.

The polyol ester preferably employed as a viscosity reducer in the process according to the invention is preferably obtainable by reaction of a monocarboxylic acid or a monocarboxylic acid derivative with a polyol. In this context, the term “monocarboxylic acid derivative” includes all derivatives of a monocarboxylic acid which lead to a corresponding polyol ester of the monocarboxylic acid in a reaction with a polyol. In particular, the term “monocarboxylic acid derivative” includes the acid chlorides of the monocarboxylic acid and the acid anhydrides of the monocarboxylic acid. These derivatives preferably have an increased reactivity of the carboxylic acid group compared with the monocarboxylic acid, so that during a reaction with a polyol the ester formation is promoted.

The polyol employed for the preparation of the polyol ester is preferably a polyol having 2 to 6 OH groups, where this can be chosen, for example, from the group consisting of ethylene glycol, propylene glycol, trimethylolpropane, glycerol, pentaerythritol, sorbitol and dipentaerythritol, the use of glycerol being particularly preferred. The monocarboxylic acid employed for the preparation of the polyester is preferably a C₁- to C₈-monocarboxylic acid, or a derivative of a C₁- to C₈-monocarboxylic acid, for example an acid chloride or an acid anhydride of a C₁- to C₈-monocarboxylic acid, particularly preferably a C₂- to C₄-monocarboxylic acid or a derivative of a C₂- to C₄-monocarboxylic acid, for example an acid chloride or an acid anhydride of a C₂- to C₄-monocarboxylic acid. Examples of suitable monocarboxylic acids which may be mentioned are, in particular, monocarboxylic acids chosen from the group consisting of formic acid, acetic acid, propionic acid, butyric acid and 2-ethylhexanoic acid, the use of acetic acid or a derivative thereof or of propionic acid or a derivative thereof being particularly preferred. The use of glycerol triacetate as the polyol ester is particularly preferred according to the invention.

The preparation of a polyol ester from a polyol and a monocarboxylic acid or from a polyol and a derivative of a monocarboxylic acid by an esterification reaction is adequately known to the person skilled in the art. Preferably, in this context the monocarboxylic acid or the derivative of the monocarboxylic acid is reacted with the polyol in an amount such that all the OH groups of the polyol are esterified. However, it is also conceivable to employ the monocarboxylic acid in an amount such that only some of the OH groups of the polyol are esterified.

According to a preferred embodiment of the process according to the invention, this can include the further process step iv) of provision of further compounds having at least two hydrogen atoms which are reactive with isocyanate groups, this process step iv) being carried out before process step iii). In principle all compounds which have at least two hydrogen atoms which are reactive with isocyanate groups and are known in connection with the preparation of polyurethanes can be employed as further compounds having at least two hydrogen atoms which are reactive with isocyanate groups. In particular, low-viscosity polyester polyols or optionally low-viscosity polyether polyols are possible here, these low-viscosity polyether or polyester polyols preferably having a viscosity determined by the Brookfield method at 25° C. of less than 500 mPas, particularly preferably of less than 250 mPas, still more preferably of less than 100 mPas and moreover preferably of less than 50 mPas.

According to a further preferred embodiment of the process according to the invention, this can also include the further process step v) of provision of further additives which differ from the components provided in process steps i), ii) and optionally iv), this process step v) also being carried out before process step iii).

In this context, all additives known to the person skilled in the art for the preparation of polyurethanes can be employed as further additives. These further additives can include, in particular, chain lengthening and/or crosslinking agents, catalysts, mould release agents, plasticizers, pore regulators, substances having a fungistatic or bacteriostatic action, dyestuffs, pigments, blowing agents, stabilizers, fillers or flame proofing agents. The amount of additives is preferably less than 25 wt. %, still more preferably less than 20 wt. % and most preferably less than 15 wt. %, in each case based on the total weight of the components provided in process steps i), ii) and optionally iv) and/or v). If fillers are employed as further additives, the amount of further additives can also be significantly higher, and under certain circumstances up to 70 wt. %, based on the total weight of the components provided in process steps i), ii) and optionally iv) and/or v).

Diols and/or triols having molecular weights of less than 400 g/mol, preferably having molecular weights in the range of from 60 to 300 g/mol, are usually employed as chain lengthening and/or crosslinking agents. Aliphatic, cycloaliphatic and/or araliphatic diols having 2 to 14, preferably 4 to 10 carbon atoms, such as, for example, ethylene glycol, 1,3-propanediol, 1,10-decanediol, o-, m- and p-dihydroxycyclohexane, diethylene glycol, dipropylene glycol and, preferably, 1,4-butanediol, 1,6-hexanediol and bis-(2-hydroxyethyl)-hydroquinone, and triols, such as 1,2,4- and 1,3,5-trihydroxycyclohexane, triethanolamine, diethanolamine, glycerol and trimethylolpropane, are, for example, possible.

Catalysts are employed, for example, in the production of rigid polyurethane foams for promoting the incorporation of isocyanurate groups. Metal carboxylates, in particular potassium acetate and solutions thereof, are conventionally employed as isocyanurate catalysts. Further catalysts which can be used for the preparation of polyurethanes are the activators known from the prior art, such as, for example, tertiary amines, tin compounds or titanium compounds.

Blowing agents are employed if polyurethane foams are to be produced. A blowing agent containing formic acid is preferably employed as the blowing agent. This can be employed as the sole blowing agent or in a mixture with water and/or physical blowing agents. Preferably, hydrocarbons, halogenated hydrocarbons, such as chlorofluorocarbons (CFCs), hydrochlorofluorocarbons (HCFCs) or hydrofluorocarbons (HFCs) and other compounds, such as, for example, perfluorinated alkanes, such as perfluorohexane, and ethers, esters, ketones and acetals or mixtures thereof, are used as physical blowing agents. In this context, hydrofluorocarbons, such as, for example, 1,1,1,3,3-pentafluorobutane, 1,1,1,3,3-pentafluoropropane, 1,1,1,2-tetrafluoroethane or 1,1,1,2,3,3,3-heptafluoropropane and mixtures thereof, are particularly preferred. Hydrocarbons, such as, for example, the isomers and derivatives of pentane, can furthermore preferably be employed as physical blowing agents.

Possible stabilizers are, in particular, foam stabilizers, antioxidants, UV stabilizers or hydrolysis stabilizers. The choice of these stabilizers depends on the one hand on the main components of the composition, and on the other hand on the application conditions and the stresses on the polyurethane to be expected. If the polyurethane is built up from polyether units in the main chain, antioxidants, optionally in combination with UV stabilizers, are chiefly necessary. Examples of these are the commercially available, sterically hindered phenols and/or thioethers and/or substituted benzotriazoles or the sterically hindered amines of the HALS (“hindered amine light stabilizer”) type. If essential constituents of the main chain of the polyurethane are made up of polyester units, hydrolysis stabilizers, for example of the carbodiimide type, are preferably employed.

Surface-active substances, i.e. compounds which serve to assist homogenization of the starting substances and are optionally also suitable for regulating the cell structure of the polyurethanes, can furthermore be employed as stabilizers. There may be mentioned, for example, emulsifiers, such as the sodium salts of castor oil sulphates or fatty acids and salts of fatty acids with amines.

Substances which promote the formation of a regular cell structure during foaming are called foam stabilizers. Examples of suitable foam stabilizers which may be mentioned are, in particular, silicone-containing foam stabilizers, such as siloxane/oxalkylene copolymers and other organopolysiloxanes. Alkoxylation products of fatty alcohols, oxo alcohols, fatty amines, alkylphenols, dialkylphenols, alkylcresols, alkylresorcinols, naphthol, alkylnaphthols, naphthylamine, aniline, alkylanilines, toluidine, bisphenol A, alkylated bisphenol A and polyvinyl alcohol, and furthermore alkoxylation products of condensation products of formaldehyde and alkylphenols, formaldehyde and dialkylphenols, formaldehyde and alkylcresols, formaldehyde and alkylresorcinols, formaldehyde and aniline, formaldehyde and toluidine, formaldehyde and naphthol, formaldehyde and alkylnaphthols and formaldehyde and bisphenol A or mixtures of two or more of these, can also be employed as foam stabilizers.

The flame proofing agents known from the prior art can in general be used as flame proofing agents. Suitable flame proofing agents are, for example, brominated ethers, brominated alcohols, such as, for example, dibromoneopentyl alcohol, tribromoneopentyl alcohol and PHT-4-diol, and chlorinated phosphates, such as, for example, tris-(2-chloroethyl) phosphate, tris-(2-chloroisopropyl) phosphate (TCPP), tris-(1,3-dichloroisopropyl) phosphate, tris-(2,3-dibromopropyl) phosphate and tetrakis-(2-chloroethyl)-ethylene diphosphate, or mixtures thereof. In addition to the halogen-substituted phosphates already mentioned, inorganic flame proofing agents, such as red phosphorus, preparations containing red phosphorus, expandable graphite (expanded graphite), aluminum oxide hydrate, antimony trioxide, arsenic oxide, ammonium polyphosphate and calcium sulphate, or cyanuric acid derivatives, such as melamine, or mixtures of at least two flame proofing agents, such as ammonium polyphosphates and melamine, and optionally starch, can also be used for rendering the polyurethanes prepared according to the invention flame-resistant.

Mould release agents which can be employed are, for example, those mould release agents which are described in DE-A-1 953 637, DE-A-2 121 670, DE-A-2 431 968 or in DE-A-24 04 310. Preferred release agents are the salts, containing at least 25 aliphatic carbon atoms, of fatty acids having at least 12 aliphatic carbon atoms and primary mono-, di- or polyamines having two and more carbon atoms or amines containing amide or ester groups which have at least one primary, secondary or tertiary amino group, saturated and/or unsaturated esters, containing COOH and/or OH groups, of mono- and/or polyfunctional carboxylic acids and polyfunctional alcohols having hydroxyl or acid numbers of at least 5, ester-like reaction products of ricinoleic acid and long-chain fatty acids, salts of carboxylic acids and tertiary amines and natural and/or synthetic oils, fats or waxes.

In addition to these release agents which are mentioned by way of example and are preferably to be employed, in principle other release agents of the prior art which are known per se can also be employed in the process according to the invention, by themselves or in a mixture with the preferred release agents mentioned by way of example. These release agents which are furthermore suitable include, for example, the reaction products of fatty acid esters and polyisocyanates according to DE-A-23 07589, the reaction products of polysiloxanes containing reactive hydrogen atoms with mono- and/or polyisocyanates according to DE-A-23 56 692, esters of polysiloxanes containing hydroxymethyl groups with mono- and/or polycarboxylic acids according to DE-A-23 63 452 and salts of polysiloxanes containing amino groups and fatty acids according to DE-A-24 27 273 or DE-A-24 31 968.

Fillers, in particular fillers having a reinforcing action, which may be mentioned by way of example are silicatic minerals, for example laminar silicates, such as antigorite, serpentine, hornblendes, amphiboles, chrysotile and talc, metal oxides, such as kaolin, aluminum oxides, titanium oxides and iron oxides, metal salts, such as chalk and barite, and inorganic pigments, such as a phthalocyanine complex and glass flour.

In process step iii) of the process according to the invention, the polyisocyanate component is now brought into contact with the polyol component, optionally in the presence of the further additives provided in process step v) and optionally the further compounds having at least two hydrogen atoms which are reactive with isocyanate groups provided in process step iv), to form a polyurethane, this bringing into contact preferably being carried out by intimate mixing of the components provided in process steps i), ii), optionally iv) and optionally v). It is furthermore preferable for the mixing of the components provided in process steps i), ii), optionally iv) and optionally v) to be carried out at a temperature of less than 60° C., particularly preferably of less than 40° C.

The precise nature and manner of bringing into contact of the individual components provided in process steps i), ii), optionally iv) and optionally v) is not critical for the inventive process (with the exception of the condition that the polyether polyol component, polyester polyol component or mixture of the polyether polyol component and the polyester polyol component employed for the preparation of the polyol component is first mixed with the polyol ester for the purpose of reducing the viscosity of the polyol component), and depends in particular on whether a foamed or a non-foamed polyurethane is to be produced. An overview of the starting substances for the processes used for the preparation of polyurethanes is to be found, for example, in Kunststoffhandbuch, volume 7, “Polyurethane”, Carl-Hanser-Verlag Munich Vienna, 1st edition 1966, 2nd edition 1983 and 3rd edition 1993. In principle, however, the components can be brought into contact continuously or discontinuously, by the one-shot process or by the prepolymer process with the aid of known mixing devices.

In the industrial production of polyurethane foams, it is conventional to combine the polyol component, the further additives and optionally the further compounds having at least two hydrogen atoms which are reactive with isocyanate groups and then to mix the mixture obtained in this way with the polyisocyanate component, it being possible for all the mixing devices known to the person skilled in the art to be employed for this. The precise ratios of amounts in which in particular the polyol component and optionally the further compounds having at least two hydrogen atoms which are reactive with isocyanate groups are reacted with the polyisocyanate component depends in this context on the properties which the polyurethane aimed for is to have. However, the polyisocyanate component and the polyol component or the mixture of the polyol component and the further compounds having at least two hydrogen atoms which are reactive with isocyanate groups are conventionally brought together in an amount such that the isocyanate index is between 50 and 500. In the context of the present invention, the isocyanate index is understood as meaning the stoichiometric ratio of isocyanate groups to hydrogen atoms which are reactive with isocyanate multiplied by 100.

According to a particularly preferred embodiment of the process according to the invention, however, process step iii) is carried out as a reaction injection molding process. In such a process it is preferable for the polyisocyanate component and the polyol component and optionally the further components provided in process steps iv) and/or v) to be conveyed by metering into a mixing chamber (it is also conceivable that individual components, in particular the components provided in process steps ii), iv) and v), are already mixed with one another before being fed into the mixing chamber) and are mixed in the mixing chamber to give a polyurethane reaction mixture, and the polyurethane reaction mixture is then discharged into the cavity of a mould via a runner. Such a process is described, for example, in DE-A-10 2004 006 074.

In this connection, it may prove to be advantageous in particular to carry out the discharging of the polyurethane reaction mixture into the cavity under a pressure of less than 5 bar, still more preferably less than 4 bar, moreover preferably less than 2 bar, and moreover still more preferably less than 1 bar and most preferably under atmospheric pressure.

Because of the comparatively low viscosity of the reaction mixture obtained in process step iii) by bringing the components provided in process steps i), ii), optionally iv) and optionally v) into contact, this can also be injected into cavities of small total volume or into cavities which include defined sections of small section volume. In this connection, it is preferable in particular for the cavity to have a total volume of less than 15 cm³, still more preferably of less than 10 cm³ and most preferably of less than 5 cm³.

A contribution towards achieving the abovementioned objects is also made by a polyurethane which is obtainable by the process described above. Preferably, this polyurethane is a shaped article of polyurethane.

A further contribution towards achieving the abovementioned objects is made by a polyol component comprising a polyether polyol, a polyester polyol or a mixture of a polyether polyol and a polyester polyol, comprising a polyol ester of a polyol and a monocarboxylic acid as a viscosity reducer, wherein those components or compounds which have already been mentioned above as the preferred component or as preferred polyether or polyester polyols or polyol esters in connection with the process according to the invention for the preparation of a polyurethane are preferred as the polyol component comprising a polyether polyol, a polyester polyol or a mixture of a polyether polyol and a polyester polyol and as the polyol ester. According to a particular embodiment of the polyol component according to the invention, this comprises the polyol ester in an amount in a range of from 0.1 to 30 wt. %, particularly preferably in a range of from 1 to 20 wt. % and most preferably in a range of from 5 to 15 wt. %, in each case based on the total weight of the polyol component.

A further contribution towards achieving the abovementioned objects is also made by a process for the preparation of a polyol component comprising a polyether polyol, a polyester polyol or a mixture of a polyether polyol and a polyester polyol, in which a polyether polyol component, a polyester polyol component or a mixture of a polyether polyol component and a polyester polyol component is brought into contact with a polyol ester of a polyol and a monocarboxylic acid, preferably by mixing. Here also, those components or compounds which have already been mentioned as preferred polyether or polyester polyols or as polyol esters in connection with the process according to the invention for the preparation of a polyurethane are preferred as the polyol component comprising a polyether polyol, a polyester polyol or a mixture of a polyether polyol and a polyester polyol and as the polyol ester. According to a particular embodiment of the process according to the invention for the preparation of a polyol component, the polyether polyol component employed is a high-viscosity polyether polyol component, which preferably has a viscosity determined by the Brookfield method at 25° C. of at least 500 mPas, particularly preferably of at least 1,000 mPas, and most preferably of at least 2,000 mPas, where the viscosity determined by the Brookfield method at 25° C. is preferably in a range of from 500 to 12,000 mPas, still more preferably in a range of from 1,000 to 10,000 mPas and most preferably in a range of from 2,000 to 8,000 mPas. If a polyester polyol component is employed, the polyester polyol component employed is preferably a high-viscosity polyester polyol component having a viscosity determined by the Brookfield method at 25° C. of at least 1,000 mPas, particularly preferably of at least 2,000 mPas, and most preferably of at least 4,000 mPas, where the viscosity determined by the Brookfield method at 25° C. is preferably in a range of from 1,000 to 20,000 mPas, still more preferably in a range of from 2,000 to 15,000 mPas and most preferably in a range of from 4,000 to 10,000 mPas.

It is also preferable for the polyether polyol component, the polyester polyol component or the mixture of the polyether polyol component and the polyester polyol component to be based on a polyether polyol, a polyester polyol or a mixture of a polyether polyol and a polyester polyol, or to consist of this, to the extent of at least 50 wt. %, still more preferably to the extent of at least 60 wt. %, moreover preferably to the extent of at least 75 wt. %, moreover still more preferably to the extent of at least 95 wt. % and most preferably to the extent of at least 99 wt. %, in each case based on the total weight of the polyether polyol component, the polyester polyol component or the mixture of the polyether polyol component and the polyester polyol component.

In connection with the process according to the invention for the preparation of a polyol component, it is furthermore preferable for the polyol ester to be brought into contact with the polyether polyol component, the polyester polyol component or the mixture of the polyether polyol component and the polyester polyol component in an amount in a range of from 0.1 to 30 wt. %, particularly preferably in a range of from 1 to 20 wt. % and most preferably in a range of from 5 to 15 wt. %, in each case based on the total weight of polyether polyol component employed, polyester polyol component employed or mixture of polyether polyol component and polyester polyol component employed and polyol ester employed.

A contribution towards achieving the abovementioned objects is furthermore made by a polyol component comprising a polyether polyol, a polyester polyol or a mixture of a polyether polyol and a polyester polyol, which is obtainable by the process described above. A contribution towards achieving the above-mentioned object is moreover made by the use of the polyol component according to the invention or of the polyol component obtainable by the process according to the invention for the preparation of a polyol component in a process for the preparation of a polyurethane, preferably in a reaction injection molding process, as has been described above.

A further contribution towards achieving the abovementioned objects is made by the use of a polyol ester of a polyol and a monocarboxylic acid as a viscosity reducer for a polyol component comprising a polyether polyol, a polyester polyol or a mixture of a polyether polyol and a polyester polyol, where here also those compounds or components which have already been mentioned above as preferred compounds or components in connection with the process according to the invention for the preparation of a polyurethane are preferred as the polyol ester, as the polyether polyol, as the polyester polyol and as the polyol component.

The invention is now explained in more detail with the aid of non-limiting examples.

### EXAMPLES

### Example 1

### Preparation of a Polyol Component According to the Invention Based on a Polyether Polyol

10 wt. % of glycerol triacetate (obtainable from Cognis Oleochemicals GmbH, Germany) is added to 90 g of a polyether polyol of sorbitol and ethylene oxide/propylene oxide (obtainable from PCC Rokita SA, Poland under the trade name Rokopol® 551 (viscosity: 3,600 mPas)). The viscosity of the polyol component obtained in this way was 1,640 mPas.

### Example 2

### Preparation of a Further Polyol Component According to the Invention

10 wt. % of glycerol tripropionate is added to 90 g of a polyether polyol of sorbitol and ethylene oxide/propylene oxide (obtainable from PCC Rokita SA, Poland under the trade name Rokopol® 551 (viscosity: 3,600 mPas)). The viscosity of the polyol component obtained in this way was 1,450 mPas.

### Example 3

### Preparation of a Polyurethane

A polyurethane was prepared on the basis of the polyol component obtained in Examples 1 and 2 and, as a comparison example, on the basis of the Rokopol® 551 product to which a polyol ester had not been added. The following components were prepared here

### Component A:

| | |
|---|---|
| 92.0 parts by wt. | polyol component from Example 1 or 2 or |
| | pure Rokopol ® 551 product; |
| 0.15 part by wt. | triethanolamine; |
| 6 parts by wt. | 1,4-butanediol; |
| 1.8 parts by wt. | DABCO (1,4-diazabicyclo[2.2.2]octane); |
| 0.05 part by wt. | dibutyltin laurate. |

### Component B:

| | |
|---|---|
| 33 parts by wt. | 4,4′-diphenylethane-diisocyanate reacted with |
| | tripropylene glycol, having an NCO content of 23%. |

The two components A and B were mixed with one another at a characteristic number of 121 and the mixture was then foamed. It was found here that component A with the polyol component obtained in Examples 1 and 2 could be mixed with component B considerably better than a component A which was prepared with a pure Rokopol® 551 product.

### Example 4

### Preparation of a Polyol Component According to the Invention Based on a Polyester Polyol

10 wt. % of glycerol triacetate was added to the polyester polyol Edenol® 1230 (viscosity: 3,840 mPas). The viscosity of the polyol component obtained in this way was 2,317 mPas.

A polyurethane was also prepared according to Example 3 with this polyol component. Here also it was found that a component A which contains the Edenol® 1230 product to which glycerol triacetate has been added could be mixed with component B considerably better than the corresponding component A with the pure Edenol®1230 product.

1. A process for the preparation of a polyurethane, comprising the process steps of:
i) providing a polyisocyanate component comprising at least one polyisocyanate;
ii) providing a polyol component selected from at least one polyether polyol, one polyester polyol, or a mixture of a polyether polyol and a polyester polyol, wherein the polyol component comprises a polyol ester of a polyol and a monocarboxylic acid; and
iii) bringing of the polyisocyanate component into contact with the polyol component to form a polyurethane.

2. The process according to claim 1, wherein process step iii) is carried out as a reaction injection molding process.

3. The process according to claim 2, wherein the polyisocyanate component and the polyol component are conveyed by metering into a mixing chamber and are mixed in the mixing chamber to give a polyurethane reaction mixture, and the polyurethane reaction mixture is then discharged into the cavity of a mold via a runner.

4. The process according to claim 3, wherein the discharge of the polyurethane reaction mixture into the cavity is carried out under a pressure of less than 5 bar.

5. The process according to claim 3, wherein the cavity has a volume of less than 15 cm³.

6. The process according to claim 1, wherein the polyether polyol is obtainable by reaction of an alkylene oxide with water, an amine, an amino alcohol, or an alcohol.

7. The process according to claim 6, wherein the alkylene oxide is ethylene oxide or propylene oxide.

8. The process according to claim 7, wherein the alcohol is an alcohol having at least 3 hydroxyl groups in the molecule.

9. The process according to claim 8, wherein the alcohol is chosen from the group consisting of trimethylolpropane, glycerol, pentaerythritol and sugar compounds.

10. The process according to claim 6, wherein the amine is an amine having at least two primary amino groups in the molecule.

11. The process according to claim 10, wherein the amine is chosen from the group consisting of phenylenediamine, 2,3-toluoylenediamine, 2,4-toluoylenediamine, 3,4-toluoylenediamine, 2,6-toluoylenediamine, 4,4′-diaminodiphenylmethane, 2,4′-diaminodiphenylmethane, 2,2′-diaminodiphenylmethane, 1,2-ethylenediamine, 1,3-propylenediamine, 1,4-butylenediamine, 1,6-hexylenediamine, 1,8-octylenediamine, diethylenetriamine and dipropylenetriamine.

12. The process according to claim 1, wherein the polyether polyol has a functionality of from preferably 3 to 8.

13. The process according to claim 1, wherein the polyether polyol has a hydroxyl number in a range of from 50 mg of KOH/g to 1,200 mg of KOH/g.

14. The process according to claim 1, wherein the polyester polyol is obtainable by condensation of polyfunctional alcohols with polyfunctional carboxylic acids.

15. The process according to claim 14, wherein the polyfunctional alcohol is a diol having 2 to 12 carbon atoms.

16. The process according to claim 14, wherein the polyfunctional carboxylic acid is a polyfunctional carboxylic acid having 2 to 12 carbon atoms.

17. The process according to claim 14, wherein the polyester polyol has a hydroxyl number in a range of from 50 mg of KOH/g to 1,200 mg of KOH/g and the polyester polyol has a hydroxyl number in a range of from 20 mg of KOH/g to 500 mg of KOH/g.

18. The process according to claim 1, wherein the polyol component is obtainable by mixing a polyether polyol component, a polyester polyol component or a mixture of a polyether polyol component and a polyester polyol component with the polyol ester.

19. The process according to claim 18, wherein the polyether polyol component has a viscosity determined by the Brookfield method at 25° C. of at least 500 mPas.

20. The process according to claim 18, wherein the polyester polyol component has a viscosity determined by the Brookfield method at 25° C. of at least 1,000 mPas.

21. The process according to claim 1, wherein the polyol component comprises the polyol ester in an amount in a range of from 0.1 to 30 wt. %, based on the total weight of the polyol component.

22. The process according to claim 21, wherein the polyol component comprises the polyol ester in an amount in a range of from 5 to 15 wt. %, based on the total weight of the polyol component.

23. The process according to claim 1, wherein the polyol employed for the preparation of the polyol ester is a polyol having 2 to 6 OH groups.

24. The process according to claim 23, wherein the polyol employed for the preparation of the polyol ester is a polyol chosen from the group consisting of ethylene glycol, propylene glycol, trimethylolpropane, glycerol, pentaerythritol, sorbitol and dip entaerythritol.

25. The process according to claim 1, wherein the monocarboxylic acid employed for the preparation of the polyol ester is a C₁- to C₈-monocarboxylic acid.

26. The process according to claim 25, wherein the monocarboxylic acid is chosen from the group consisting of formic acid, acetic acid, propionic acid, butyric acid and 2-ethylhexanoic acid.

27. The process according to claim 1, wherein the polyol ester is glycerol triacetate or glycerol tripropionate.

28. A polyurethane obtainable by the process according to claim 1.

29. A polyol component comprising at least one polyether polyol, one polyester polyol or a mixture of a polyether polyol and a polyester polyol, comprising a polyol ester of a polyol and a monocarboxylic acid.

30. A process for the preparation of a polyol component comprising a polyether polyol, a polyester polyol or a mixture of a polyether polyol and a polyester polyol, in which a polyether polyol component, a polyester polyol component or a mixture of a polyether polyol component and a polyester polyol component is mixed with a polyol ester of a polyol and a monocarboxylic acid.

31. A polyol component comprising a polyether polyol, a polyester polyol or a mixture of a polyether polyol and a polyester polyol, obtainable by the process according to claim 30.

32. Use of the polyol component according to claim 29 as compounds having at least two hydrogen atoms which are reactive with isocyanate groups in a process for the preparation of a polyurethane.

33. Use of a polyol ester of a polyol and a monocarboxylic acid as a viscosity reducer for a polyol component comprising a polyether polyol, a polyester polyol or a mixture of a polyether polyol and a polyester polyol.
